(19) United States
(12) Patent Application Publication
(10) Pub. No.: US 2014/0057007 A1
(43) Pub. Date: Feb. 27, 2014

(54) Food, particularly a beverage for human consumption

(71) Applicant: Marco Zipoli, Genova (IT)

(72) Inventor: Marco Zipoli, Genova (IT)

(21) Appl. No.: 14/113,446

(22) PCT Filed: Apr. 27, 2012
(86) PCT No.: PCT/IB2012/052112

§ 371 Date: Nov. 7, 2013

(30) Foreign Application Priority Data
Apr. 29, 2011 (IT) GE2011A000050

(51) Int. Cl. A61K 31/4015; A61K 31/198; A61K 31/205; A61K 31/197; A23L 2/52; A61K 36/16

(52) U.S. Cl. CPC A61K 31/4015; A23L 2/52; A61K 36/16; A61K 31/205; A61K 31/197; A61K 31/198
USPC 424/752; 514/424

(57) ABSTRACT

Food, particularly a beverage for human consumption, the ingestion of which enhances cognitive processes such as intelligence, memory, creativity and mood, and/or produces energizing effects. Such food is composed of a composition dilutable or diluted in a liquid or semi-liquid solution. The composition includes at least one nootropic substance, preferably a cholinergic nootropic substance, that is, a substance involved in the formation and/or functioning of the acetylcholine neurotransmitter in combination with L-theanine

### FIELD OF THE INVENTION

The present invention relates to a food, particularly a beverage for human consumption, the ingestion of which enhances cognitive processes and/or produces energizing effects.

The present invention relates also to a dilutable composition and/or a liquid or semi-liquid solution for producing such food, a process method for said liquid or semi-liquid solution and a kit for administering such food and for monitoring the effects due to the ingestion of the substances contained in said food.

### BACKGROUND OF THE INVENTION

It is known that the functioning of the central nervous system requires the use of neurotransmitters.

The fact of increasing or keeping the balance in the concentration of neurotransmitters (acetylcholine, dopamine, norepinephrine, serotonin) allows mental performances to be kept high.

Substances involved in the formation of neurotransmitters are taken through the diet.

For instance choline, which is found in pulses, cereals, and egg yolk, is essential for the synthesis of acetylcholine neurotransmitter and of the main components of cell membranes, phosphatidylcholine and sphingomyelin.

Nutritional supplements of choline are important for the prevention of many pathologic conditions, and are used for treating or preventing several human diseases, such as arteriosclerosis and some deficiencies in brain and memory functioning.

It has also been proved that choline is essential for the proper development of the brain in infants and children. This important supplement does not allow acetylcholine levels to be reduced, which reductions are associated to memory loss and to learning difficulties, which mainly occur with aging.

Acetylcholine is a fundamental substance for many cerebral processes such as memory, concentration, calculation and creativity. Increasing the concentration of this neurotransmitter allows brain functions to be enhanced.

It is known to use substances defined as nootropic substances which enhance human cognitive processes.

Several nootropic substances are contained in foods or plant parts (herbs, roots, beans, barks) or are drugs used for treating people with learning difficulties or degenerative diseases of the central nervous system.

Nootropic substances are involved in the formation of neurotransmitters in order to enhance concentration, calculation ability, memory, creativity and mood.

Nootropic substances have a very low toxicity, with few side effects and allow the brain aging to be slowed down, in addition to the fact that they can be even able to improve the brain functioning, in people with cognitive functions defined as “normal”.

Nootropics fall within the safest drugs as regards toxicology and side effects are restricted to excitability, headache, nausea, agitation, insomnia and anxiety, disorders that in most cases are due to an overdosage.

Nootropics improve brain metabolism, stimulating oxidative catabolism, increasing energy levels (ATP and cAMP), increasing phospholipid metabolism and the protein biosynthesis. Moreover they seem to have an impact on hippocampal acetylcholine and dopamine release.

Nootropics improve, enhance and sensitize receptor activities, such to allow the brain aging process to slow or reverse.

It is also known to use plant extracts having energizing or stimulant effects.

Ginkgo biloba is particularly known for its action on venous, arterial and above all cerebral circulation: it protects arteries, veins and capillary vessels from being damaged, it helps in regulating their tone and their elasticity, it improves the peripheral circulation, it helps in distributing oxygen and glucose to the brain.

Ginkgo biloba is a plant whose active principles contained into the leaves are used for improving the supply of oxygen to the brain: by increasing the supply of oxygen to the brain the mental acuity, concentration, short-term memory and cognitive ability improve.

Therefore it can be used for improving memory, consciousness condition in elderly people and blood circulation in general.

It is also used for slowing the progression of Alzheimer's disease, in addition to having properties inhibiting 5-alpha reductase. Ginkgo biloba as antioxidant protects the brain and nervous system from damages caused by free radicals, and this can help in controlling the aging effects.

Ginkgo biloba leaves contain:

terpenes, ginkgolide B with an action inhibiting Paf-acether, intercellular phospholipid mediator involved in the platelet. aggregation, in thrombus formation, atherogenesis and in capillary hyperpermeability;

polyphenols, flavonoids (ginketolo, isiginketolo, bilabetolo, ginkolide), with activity on all the levels of the circulatory system: vasodilatative activity on arteries, increase of venous tone, increase of cerebral circulation; ginketolo, isiginketolo and bilabetolo act on cell membranes, stabilizing them, particularly ginkolide blocks the lipid peroxidation and the formation of free radicals and it inhibits platelet activating factor (PAF) consequently reducing blood viscosity.

Guayusa (Ilex guayusa) is a plant native to the Amazon Rainforest whose leaves are used for preparing beverages increasing physical and mental abilities, such as memory and mental clarity, and they improve mood.

Other effects are also known such as: analgesic, expectorant, positive action on digestion and peristalsis, diuretic, stimulant of mammary glands, aphrodisiac, regulator of blood glycemic index.

These effects are due to alkaloids provided into the leaves such as: caffeine (one cup of guayusa contains 90 mg of caffeine), theophylline (present also in green tea), theobromine (present even in cacao and in its derivatives and, in small amounts in tea leaves). The plant contains also L-theanine amino acid that has calming effects on the nervous system and protects and restores nervous components.

Guayusa extract further contains antioxidants (50% more than green tea), polyphenols (one cup of guayusa has 30% more polyphenols than a cup of green tea which stimulate the immune system, with anti-inflammatory and anti-tumor activity), flavonoids, saponins (which have beneficial effects on blood cholesterol levels, cancer, bone health and on the stimulation of the immune system), amino acids, minerals, vitamins.

Guarana (Paullinia cupana) is an evergreen climbing plant native to Amazon Rainforest used for the tonic stimulant effect of the substances contained in the seeds. Seeds contain guaranine, similar to caffeine. This active principle is widely used as stimulant, both in extemporaneous preparations and in the form of specific nutritional supplements or energy beverages.

In the sport field guarana extracts are successfully used for improving both physical and mental endurance.

It is often recommended as a supplement useful for losing weight faster and for suppressing appetite.

Yerba mate (Ilex paraguariensis) is a plant native to South America from the leaves of which a drink is obtained that is very popular in South America.

Yerba mate contains, in addition to minerals such as potassium, magnesium and manganese, natural alkaloids such as caffeine (the main element whose content ranges from 0.7% to 1.7% of dry weight compared with 0.4-9.0% for tea leaves, 2.5-7.6% in guarana, and up to 3.2% for coffee), theobromine (content ranging from 0.3% to 0.9%) and theophylline.

Studies about yerba mate have shown that the xanthine cocktail therein is different from other plants containing caffeine, since it has positive effects on muscle tissue and on the central nervous system. Therefore it has been found that the synergy of the three xanthines present in mate has a relaxing effect on smooth muscle tissue and a stimulant effect on myocardial (heart) tissue.

Other studies have shown that it can help in lowering cholesterol, reducing the tendency to obesity and it has a high antioxidant capacity.

The plants mentioned above contain alkaloids such as caffeine, theobromine, theophylline (methylxanthines) and L-theanine (amino acid derivative of L-glutamic acid contained in guayusa).

Recent studies have shown that caffeine could have nootropic effects, inducing some changes in memory and learning.

Caffeine further considerably reduces the risk of heart disease.

L-theanine has psychoactive properties. It has been shown that L-theanine drastically reduces mental and physical stress, it further improves cognition and mood. L-theanine can help the body in the immune response to infections, by stimulating the disease fighting capacity of gamma delta T cells.

In particular L-theanine, a molecule with an amino acid structure generally found in tea leaves, is appreciated both as flavouring agent, since it reduces the bitter taste of tea, and as a relaxing and anti-stress substance.

It is known that L-theanine can serve the following functions:

relaxing action,

antagonist action against caffeine,

improvement of learning and concentration abilities,

lowering blood pressure,

relations with dopamine, serotonin, and other neutrotransmitters,

modulation of premenstrual syndrome symptoms,

modulation of immune system activity,

modulation of anti-tumor therapy.

Theobromine has an effect similar to caffeine on the human nervous system, even if at a lower extent.

Theobromine is used as a vasodilator, as a diuretic and as heart stimulant. Moreover, theobromine can be used also for cancer prevention.

Theophylline is a methylxanthine used in the therapy for respiratory diseases.

Theophylline leads to relaxation of bronchial smooth muscle, increases heart muscle contractility and efficiency, increases blood pressure, increases the supply of blood to kidneys, has an anti- inflammatory action and has a stimulatory effect on the central nervous system mainly on the medullary respiratory center.

### SUMMARY OF THE INVENTION

The object of the present invention is a food, particularly a beverage for human consumption, the ingestion of which improves cognitive processes such as intelligence, memory, creativity and mood, and/or causes energizing effects.

The food is composed of a composition dilutable or diluted in a liquid or a semi-liquid solution, which composition comprises at least one nootropic substance, preferably a cholinergic nootropic substance that is a substance involved in the formation and/or functioning of acetylcholine neutrotransmitter, in combination with L-theanine

Said food is therefore composed of a composition intended to improve mental performances, which composition can be mixed into a liquid component, preferably a food liquid solution based on vegetable extracts, with energizing function.

The beverage obtained by mixing the two components, that is the energizing component and the component stimulating the nervous system, is particularly useful for sportsmen, students, creative and elderly people with or without memory problems.

The two components can be mixed together by the user upon the ingestion or can be pre-mixed in order to obtain a complete beverage ready for being sold and consumed.

### DETAILED DESCRIPTION OF EMBODIMENTS OF THE INVENTION

According to the present invention the composition intended to enhance mental performance, which appears as a food material in granular or powder form, comprises at least one nootropic substance in combination with L-theanine

One or more nootropic cholinergic substances are preferably used namely substances involved in the formation and/or functioning of the acetylcholine neurotransmitter.

As the nootropic cholinergic substance it is possible to use:

one or more nootropic substances from the racetam class (that is nootropics that have in common the nucleus of 2 -pyrrolidone), particularly piracetam,

choline, preferably in the form of choline bitartrate, choline citrate, choline chloride, choline alphoscerate and CDP choline.

Said substances can be used alone or in a mixture with one another.

An example of a substance belonging to racetam class is pramiracetam.

Preferably choline is provided in combination with vitamin B5, in order to help the conversion of choline into acetylcholine.

The synergy between choline and vitamin B5 leads to a higher production of acethylcholine neurotransmitter, thus improving mental clarity and memory.

Vitamin B5 or pantothenic acid, serving a very-important role in cell metabolism, can be also effective in treating the following diseases/disorders:

| Organs/parts of the body | Diseases |
|---|---|
| Intestinal system | Diarrhoea |
| | Flatulence |
| | Costiveness and post surgical gases |
| | Duodenal ulcer |
| | Worms |
| Articulations | Rheumatoid arthritis |
| | Articulation disorders |
| | Gout |
| Hair/Scalp | Baldness |
| | White hair |
| Brain/Nervous system | Nervous disorders |
| | Epilepsy |
| | Insomnia |
| | Mental disease |
| | Neuritis |
| | Multiple sclerosis |
| | Faintings |
| Legs | Muscle cramps |
| | Phlebitis |
| Glands | Adenal burnout |
| Muscles | Muscular distrophy |
| | Myasthenia gravis |
| Eyes | Cataracts |
| | Glaucoma |
| | Peripheral neuritis |
| Bones | Bone disorders |
| | Fracture |
| Skin | Acne |
| | Psoriasis |
| | Wrinkles |
| Feet | Formication and burning sensation |
| Lungs/Respiratory system | Allergies |
| | Asthma |
| | Tubercolosis |
| Blood/circulatory system | Anemia |
| | Hypoglicemia |
| Stomach | Dyspepsia |
| | Gastritis |
| | Nausea |
| | Ulcer |
| Head | Headache |
| Vesica | Cystitis |
| General | Fatigue |
| | Athletic performance |
| | Cancer |
| | Depression |
| | Radiation damage |
| | Ethylism |
| | Infections |
| | Growth retardation |
| | Stress |
| | Old age spots |
| | Aging |
| | Premature old age |
| | Immune system |

Pantothenic acid or vitamin B5 increases energy, improves athletic performances, slows the aging process and it helps in keeping stress under control, an important factor for heart patients.

Preferably the composition intended to improve mental performances comprises piracetam in combination with L-theanine

Piracetam is a substance having the following advantages:

it improves attention, cooperation, socialization and intelligence quotient,

it stimulates a greater desire to learn, it improves concentration and intelligence.

it improves mental performances and preserves memory,

it improves reading and accuracy in cases of dyslexia, such as reading speed, writing and orthography,

it improves or slows the deterioration in senile dementia such as Alzheimer's disease.

it reduces the seriousness and the presence of headache symptoms, particularly those related to brain concussion.

Piracetam is a cholinergic agent used also for treating movement and vertigo diseases and for treating alcoholism.

A preferred embodiment of the present invention provides the two nootropic substances mentioned above that is piracetam and choline, with or without vitamin B5, to be provided in combination to one another.

Piracetam has a synergistic effect with choline, which has a synergistic effect with vitamin B5.

It has been noted that, in the beverage of the invention, the combined use of L-theanine with piracetam allows the effect of the nootropic substance to be extended and to be kept constant over time.

According to a variant of the present invention it is possible to use in combination or as an alternative to piracetam the extract of Ginkgo biloba.

It is possible to provide said food composition to comprise even one or more food excipients and/or other ingredients in order to improve nutritional properties and/or organoleptic properties.

In particular it is possible to provide to add to one or more nootropic substances mentioned above:

sweeteners such as dextrose, sucralose, stevioside and/or rebaudioside A

flavourants and/or flavour modifiers,

preservatives,

coloring agents

antioxidants

mineral supplements such as calcium carbonate/citrate, magnesium carbonate, oligoelements.

prebiotic, probiotic, energy vitamin supplements,

stabilizers, thickeners, texture modifiers such as lecithins, agar-agar, Guam seed flour, starches, pectins, carrageenans.

Therefore other components can be provided such as vitamins, mineral salts, carnosine, carnicine, vegetable extracts or other food-grade substances.

A dilutable composition of the present invention for preparing one liter of a beverage can contain:

nootropic substance of the racetam class, particularly piracetam: from 0.5 to 10 g/l, preferably 3 g/l,

L-theanine: from 200 to 1000 mg/l, preferably 400 mg/l,

choline (in one or more of the following forms: choline bitartrate/choline citrate/choline chloride/choline alphoscerate/CDP choline): from 50 to 2000 mg/l, preferably 1500 mg/l,

vitamin B5: from 0 to 2000 mg/l, preferably 10-300 mg/l.

Said substances, in particular the substance of the racetam class and choline, can be provided in the composition constituting the food, alone or in a mixture with one another.

The composition can further contain alternatively to one another:

dextrose: from 0 to 8 g/l, preferably 6 g,

sucralose: from 0 to 250 mg/l, preferably 45 mg/l,

stevioside or rebaudioside A: from 0 to 1 g/l, preferably 150 mg/l.

A preferred composition for preparing 3.14 dl of a beverage is as follows:

piracetam: 1 g

L-theanine: 133 mg

choline (in one or more of the following forms: choline bitartrate/choline citrate/choline chloride/choline alphoscerate/CDP choline): 450 mg

vitamin B5: from 10 to 300 mg

The composition can further contain alternatively to one another:

dextrose: 2 g

sucralose: 31 mg

stevioside or rebaudioside A: 48 mg.

The amounts mentioned above can be also 10 times greater without leading to toxicity symptoms for the body.

According a variant it is possible to provide in combination or instead of a nootropic substance mentioned above the vegetable extract of Ginkgo biloba.

A composition of the present invention for preparing one liter of a beverage can contain, in combination with or instead of one or more nootropic components mentioned above, from 100 to 250 mg/l of Ginkgo biloba extract, preferably 200 mg/l.

A solid composition for producing 3.14 dl of beverage can contain:

piracetam: 1 g

1-theanine: 133 mg

choline (in one or more of the following forms: choline bitartrate/choline citrate/choline chloride/choline alphoscerate/CDP choline): 450 mg-Vitamin B5: from 10 to 300 mg

Ginkgo biloba: 60 mg.

The composition can further contain alternatively to one another:

dextrose: 2 g

sucralose: 15 mg

stevioside or rebaudioside A: 48 mg.

Moreover such substances have synergistic effects one with respect to the other: if they are combined together they considerably increase their properties.

The synergy of choline, vitamin B5, Piracetam and, if provided, Ginkgo biloba and L-theanine leads to several advantages such as:

a greater production of acethylcholine neurotransmitter, which improves mental clarity and memory,

a better vascular function due to Ginkgo biloba, which therefore improves the supply of blood to the brain,

due to piracetam, an overall increase of cognitive performances of the individual.

Due to the presence of L-theanine the effect of the substances having the function of stimulating mental functions is extended over time and is kept constant for all the time the substances are present in the body.

The composition of the present invention is preferably appears as a powder mixture or soluble granules to be added and mixed by a shaking action to a liquid or semi -liquid composition.

The solid component of the present invention is able to completely dissolve into the liquid without forming suspensions or leaving any residues on the walls or bottom of the container.

The composition described above can be pre-mixed, when preparing the beverage, before selling it, with a liquid or a semi-liquid substance and sold in the liquid or semi-liquid form.

As an alternative the composition described above can be kept separated from the liquid component up to the ingestion of the beverage: therefore it is up to the user to mix the solid component and the liquid or semi -liquid component, that together compose the beverage of the present invention, such that properties and organoleptic characteristics of the substances constituting the food ^{J} are preserved and extended over time.

It is possible to provide said composition to be provided also as a gel, by adding gelling agents to the mixture mentioned above, which gel can be taken directly or after being mixed with a liquid.

As the liquid component for preparing the beverage it is possible to use spring natural water, oligomineral water, mineral water, fruit juices or the like.

Object of the present invention is also to a liquid or semi -liquid solution to be used as a beverage or for preparing a beverage enhancing cognitive processes and/or having energizing effects.

According to a preferred embodiment of the present invention the food is composed of a liquid or semi-liquid solution that is used for dissolving and diluting the component comprising one or more nootropic substances and L-theanine as described above, such to obtain a stimulant and energizing beverage.

Therefore an object of the present invention is a food and/or even a liquid or semi-liquid solution comprising plant extracts containing flavonoids, saponins, amino acids, such as L-thanine and xanthines (caffeine, theobromine, theophylline), in particular a liquid solution containing, in combination or as an alternative to one another, extracts of guayusa (Ilex guayusa), gurana (Paullinia cubana), and/or yerba mate (Ilex paraguariensis).

A liquid solution of the present invention for preparing one liter of a beverage can comprise dried parts of at least one of the following plants:

guayusa: from 5 to 40 g/l, preferably 15 g/l

guarana: from 2 to 20 g/l, preferably 8 g/l

yerba mate: from 3 to 20 g/l, preferably 10 g/l.

The solution can further contain alternatively to one another:

dextrose: from 0 to 30 g/l, preferably 20 g/l,

sucralose: from 0 to 250 mg/l, preferably 180 mg/l,

stevioside and/or rebaudioside A: from 0 to 700 mg/l, preferably 270 mg/l.

Preferably the liquid solution of the present invention contains at least one extract from plants belonging to Ilex genus.

The extract of guayusa leaves contain a mixture of alkaloids, such as caffeine, theophylline, theobromine, and L-theanine, which generate a balanced energizing effect without nervous crashes and tachycardia.

These three extracts mixed together in suitable proportions produce a very strong synergistic action by enhancing individual performances. For instance it has been proved that L-theanine, present in guayusa plant, drastically reduces mental and physical stress and it improves cognition and mood in a synergistic manner with caffeine.

In particular the liquid solution contains a mixture of alkaloids such as caffeine, theophylline, theobromine and L-theanine amino acid allowing a balanced energizing effect without nervous crashes and tachycardia to be obtained.

Obviously it is possible to provide said liquid or semi-liquid food solution to comprise even one or more food excipients and/or other ingredients in order to improve nutritional properties, organoleptic properties, as provided for the dilutable composition mentioned above.

The ingestion of a liquid or semi -liquid composition comprising extracts of said plants, in combination or alternatively to one another, particularly extracts of guayusa (Ilex guayusa), guarana (Paullinia cubana) and/or yerba mate (Ilex paraguariensis):

helps in keeping oneself young,

stimulates physical and mental performances,—it has an excellent “anti-fatigue” effect,

it is an effective analgesic,

acts as expectorant,

acts as a diuretic,

helps in regulating the menstrual cycle,

has aphrodisiac effects,

stimulates memory and mental clarity,

regulates the glycemic level in the blood,

acts as a strong antioxidant,

helps in feeling replete therefore allowing weight to be reduced.

A preferred embodiment of the present invention provides the liquid or semi-liquid solution to contain extracts from all the three plants.

Said liquid or semi-liquid solution can be used as a liquid component to be mixed with the food composition comprising at least one nootropic substance and L-theanine, as described above, for producing a food product, in particular a beverage for human consumption.

The dilution of said food composition comprising one or more nootropic supplements and L-theanine to be diluted in the aqueous solution of the present invention, further improves the advantages deriving from the ingestion of the beverage.

The ingestion of the beverage:

improves attention, cooperation, socialization and intelligence quotient,

stimulates a greater desire to learn, improves concentration and intelligence,

stimulates mental performances and preserves memory even during aging process,

increases the production of acethylcholine neurotransmitter,

increases the supply of blood to the brain,

improves reading ability and the accuracy in cases of dyslexia, such as reading speed, writing and orthography.

improves or slows the deterioration in senile dementia such as Alzheimer's disease.

reduces the seriousness and the presence of headache symptoms,

it can be successfully used for treating movement and vertigo diseases

it can be successfully used for treating alcoholism.

Said beverage has also low calories therefore it is suitable for being taken even during slimming diets and weight maintenance diets.

In particular the beverage obtained by mixing the component, in powder or granules, comprising one or more nootropic substances and L-theanine, with the liquid component, that is the liquid or semi -liquid solution with one or more vegetable extracts containing xanthines allows a beverage to be obtained able to stimulate intelligence, creativity and memory and at the same time it allows an energizing beverage to be obtained that is able to give more strength, more physical endurance, and able to stimulate metabolism, allowing fat to be burnt.

In particular due to the provision of L-theanine the beverage of the present invention combines stimulating effects of nootropic substances on the central nervous system with energizing effect of vegetable extracts reducing the drawbacks deriving from taking such substances: L-theanine reduces hyperexcitability, headache, pressure increase, tachycardia caused by stimulant substances such as caffeine and at the same time it cooperates with nootropic substances, extending the effect and keeping such stimulant effect constant over time.

Object of the present invention is also a method for producing the aqueous solution comprising one or more vegetable extracts to be used as a beverage or preferably for producing a beverage for human consumption.

The method for preparing the aqueous solution containing vegetable extracts provides to select one or more plants containing the active principles (amino acids such as L-theanine and xanthines such as theophylline, theobromine and caffeine) necessary for preparing the liquid or semi-liquid solution that can be used for preparing the beverage.

Each plant is dried under controlled atmosphere and the dried material is ground.

The extraction of the active principles contained in each plant is made by known ultrasound extraction machines. The plant uses an extraction system with a solvent, for example demineralized water, and ultrasounds which allows cells of the individual leaves to be broken and the compounds to pass into the solvent, guaranteeing a rapid extraction of the active principles (about 40 minute cycle).

Once the active principles of the individual plants are obtained in the form of a liquid, since they are dissolved into a solvent, preferably water, said liquids are mixed together in order to obtain a solution enriched with the active principles contained in the selected plants and usable for producing the liquid solution or the beverage of the present invention.

In order to facilitate the transport the liquid solutions obtained from each plant or the mixture of said solutions can be further treated in a spray-dryer used for spraying and drying liquid suspensions. The machine allows dried particles to be obtained from the liquid suspension or suspensions.

As an alternative the liquid solutions obtained from each plant or the mixture of said solutions can be subjected to a freezing process at a temperature very lower than 0° C. preferably at −42 ° C., such to obtain cakes or blocks of frozen liquid containing the active principles of interest.

One or more nootropic substances, L-theanine, sweeteners or other components can be added to the liquid solution or to the mixture of concentrated liquid solutions with active principles, which can enrich the content, improve nutritional properties, improve the color, taste or scent.

According to a preferred solution dextrose or sucralose, or stevioside or rebaudioside A is added as the sweetener to the liquid solution or to the mixture containing the active principles extracted from the plant or plants.

Generally for producing one liter of aqueous solution 280 mg of sucralose or 20 g of dextrose or 420 mg of stevioside or rebaudioside A are used.

Therefore a concentrated liquid solution is obtained containing the active principles such as amino acids such as L-theanine and xanthines extracted from one or more energizing plants and therefore containing the active principles contained in the aerial parts of said plants.

Extraction steps followed by the spray-dryer treating steps or freezing steps allow active principles contained in the used plants to be concentrated and thus allow bottling, transport and marketing costs of the product to be reduced.

Obviously it is also possible to provide said solution to be in a semi-liquid form for instance by providing the addition of thickeners or by using a liquid with a higher density instead of water.

The liquid or semi -liquid concentrated solution can be diluted by adding water or another liquid with a higher density such as yoghurt, milk, fruit- concentrate or the like.

Generally the dilution provides a dilution of 10 to 1 of water to solution or mixture described obtained by the method described above in order to obtain a beverage to be drunk.

Obviously the process described above can be used for obtaining an aqueous solution comprising only one or more of the vegetable extracts set forth above that is only one or two extracts selected among yerba mate, guarana and guayusa.

The concentrated or diluted liquid solution can be taken directly as an energy beverage or can be used for diluting and dissolving the food composition based on one or more nootropic substances described above in order to obtain a beverage that due to the synergy of the active principles contained in the dilutable component and in the liquid component, improves cognitive processes and has energizing effects.

The synergistic action found between piracetam, and L-theanine, contained also in the used vegetable extracts, on a sample of 30 test users has shown about 10% increase of the enhancement of intellectual performances and creativity with respect to the same number of users that have used the two components separately, namely the liquid solution with vegetable extracts and the dilutable composition comprising at least one nootropic substance.

This enhancement is due to the greater supply of acethylcholine neutrotransmitter in the hippocampal area and to the increase of the communication through the corpus callosus, allowing the two cerebral hemispheres to communicate in a better way. This synergy is able to improve the communication through this neuronal network and so to improve inter-hemisphere contacts. It has been deduced that this represents the possibility of having new ideas (the right hemisphere is the artistic and creative one) and of putting them into practice more rapidly (the left hemisphere is the logical and analytical one). In other words, it is an effective manner to connect the Yin and Yang parts of the cerebellum.

The test has been carried out by software by the Cogstate company.

Cogstate Research is a cognitive testing information system specific for clinical studies having a proven validity, reliability and sensitivity. Its efficacy has been proved in a great amount of international clinical studies carried out by some of the most important pharmaceutical companies. This software can be used by the users of the beverage in order to test the cognitive progresses when using it; the software is flexible, easy to be used and it generates clear graphs that can be consulted by the individual users.

The present invention relates also to a kit for administering substances stimulating intelligence, creativity and memory and/or energizing substances and for controlling the effects of said substances on the user, which kit provides at least one container for solid or liquid substances having a stimulant and/or energizing effect and means for controlling and monitoring the effects deriving from the ingestion of said substance or substances.

In particular the kit provides:

at least a container containing a dilutable composition for preparing a product for human consumption, which composition comprises at least a nootropic substance and L-theanine as described above,

at least one container containing a liquid or semi-liquid solution for the dilution of said composition,

means for controlling and monitoring the effects resulting from the ingestion of the dilutable composition and/or of the liquid or semi -liquid solution, said containers being provided in combination or alternatively to one another.

Preferably the liquid or semi-liquid solution is composed of a solution comprising one or more vegetable extracts of tropical plants, particularly a liquid or semi-liquid solution containing, in combination or alternatively to one another, extracts from guayusa (Ilex guayusa), guarana (Paullini cubana) and/or yerba mate (Ilex paraguariensis), as described above.

According to one embodiment as an alternative to said containers containing the dilutable composition and the liquid solution it is possible to provide a single container containing the beverage obtained by mixing the composition comprising at least one nootropic substance and L-theanine with the liquid or semi-liquid containing the extract from at least one plant of the above mentioned ones.

It is possible to provide to manufacture and market:

kits containing at least one container containing the dilutable composition for example in the form of powder or granules in combination with said means controlling and monitoring the effects,

kits containing at least one container containing the liquid or semi-liquid solution comprising one or more vegetable extracts such as guayusa, guarana and yerba mate in combination with said means controlling and monitoring the effects,

kits containing at least one container containing the dilutable composition for example in the form of powder or granules, at least one container containing a liquid or semi-liquid solution comprising one or more vegetable extracts such as guayusa, guarana and yerba mate, said containers being provided in combination with said means controlling and monitoring the effects,

kits containing at least one container containing the liquid or semi-liquid solution comprising one or more vegetable extracts such as guayusa, guarana and yerba mate the composition comprising at least one nootropic substance and L-theanine having being mixed with such solution.

According to a preferred embodiment said means controlling and monitoring the effects are composed of at least a software executing tests controlling and monitoring the nervous responsiveness, cognitive processes and/or physical and mental performances.

One example of the means evaluating the cognitive processes is described in the international patent PCT/IT2001/000542 with the title: “Method and apparatus for evaluating the cognitive performance of an individual”.

The components constituting or necessary for preparing the food of the present invention, in particular the dilutable composition comprising one or more nootropic substances and the liquid or semi -liquid solution comprising one or more vegetable extracts, can be packed into several types of containers which can be made of glass, plastic, aluminum or cardboard/aluminum/plastic (such as Tetra Pak).

Preferably a beverage containing the composition comprising at least one nootropic substance and L-theanine mixed with the liquid component containing extracts of at least one plant selected among guayusa, guarana and yerba mate is packed such that the beverage is ready to be drunk.

As an alternative the two components are separately packed such that the dilutable component is mixed with the liquid or semi-liquid component only upon the ingestion.

This guarantees that the substances composing the beverage keep their properties unchanged up to the ingestion moment.

This separation also allows the user to select, on the basis of his/her needs, the amount of nootropic substances to be diluted in the liquid solution.

An embodiment of the invention provides the liquid or semi -liquid solution comprising vegetable extracts to be contained into a bottle closed by a storage cap provided inside it, coinciding with the bottle opening, with a chamber containing the component to be diluted.

According to an embodiment by turning the cap on the bottle neck or by acting on the top of the cap made like a plunger, a membrane separating the dilutable component from the liquid is broken, which membrane is provided for closing the internal chamber of the cap or, depending on the type of cap used, the chamber containing the component to be diluted falls within the bottle.

The breaking of the membrane or the falling of the chamber, consequently causes the composition to be diluted to fall within the liquid. The following mixing, by a shaking action, allows the beverage object of the present invention stimulating mental processes and/or having energizing effects to be obtained.

Examples of storage caps are the single-dose system caps by Bormioli Rocco available on the web site www.bormioliroccoplastics.com.

Obviously it is possible to provide the container intended to store the beverage to contain both the dilutable component comprising nootropic substances and the liquid component with vegetable extracts mixed together.

Below some embodiments of the present invention are shown which however have not a limitative sense.

Example of a composition of the liquid component for preparing one liter of the beverage.

Guayusa: preferably 15 g/l

Guarana: preferably 8 g/l

Yerba Mate: preferably 10 g/l

Sucralose: preferably 180 mg/l or dextrose, preferably 20 g/l or stevioside or rebaudioside A preferably 270 mg/l.

Example of a composition of the powder component for preparing a 3.14 dl beverage.

Choline bitartrate: preferably 450 mg

Vitamin B5: preferably 6 mg

Piracetam: preferably 1 g

(Ginkgo biloba: 60 mg)

L-theanine: 133 mg

Dextrose: preferably 2 g

Sucralose: preferably 31 mg

Stevioside or rebaudioside A: preferably 48 mg/l.

The invention claimed is:

1. A food, particularly a beverage for human consumption, an ingestion of which enhances cognitive processes such as intelligence, memory, creativity and mood, or produces energizing effects, comprising:
a composition dilutable or diluted in a liquid or semi-liquid solution, said composition comprising at least one nootropic substance, in combination with L-theanine

2. The food according to claim 1, wherein said composition comprises:
said nootropic substance from the racetam class and choline, said nootropic substance and choline being provided alternatively to one another or in a mixture.

3. The food according to claim 2, wherein said choline is provided in combination with vitamin B5 (pantothenic acid).

4. The food according to claim 1, wherein said composition comprises, in combination with or in alternative to said nootropic substance, a vegetable extract of Ginkgo biloba.

5. The food according to claim 1, wherein said composition comprises dextrose, sucralose, stevioside, or rebaudioside A as a sweetener.

6. The food according to claim 1, wherein said composition comprises:
from 0.5 to 10 g/l of piracetam;
from 50 to 2000 mg/l of choline in one or more of the following forms: choline bitartrate, choline citrate, choline chloride, choline alphoscerato, or CDP choline;
from 0 to 2000 mg/l of vitamin B5; and
from 200 to 1000 mg/l of L-theanine;
one of the following compounds being provided as a sweetener:
from 0 to 8 g/l of dextrose;
from 0 to 250 mg/l of sucralose;and
from 0 to 1 g/l of stevioside or rebaudioside A.

7. The food according to claim 1, wherein for preparing 3.14 dl of a beverage said composition comprises:
piracetam: 1 g;
L-theanine: 133 mg;
choline in one or more of the following forms: choline bitartrate, choline citrate, choline chloride, choline alphoscerato, or CDP choline: 450 mg;
vitamin B5: from 10 to 300 mg; and
one of the following sweeteners:
dextrose: 2 g;
sucralose: 31 mg; or stevioside or rebaudioside A: 48 mg.

8. The food according to claim 4, wherein, for preparing one liter of a beverage, said composition comprises from 100 to 250 mg/l of vegetable extract of Ginkgo biloba, for 3.14 dl of the beverage.

9. (canceled)

10. The food according to claim 1, wherein said composition is provided as a mixture in soluble granules or powder to be added and mixed by a shaking action to a liquid or semi-liquid solution, and wherein the liquid or semi-liquid solution comprises vegetable extracts of plants containing flavonoids, saponins, or amino acids. and xanthines

11. The food according to claim 1, wherein said composition is a dilutable composition or a liquid or semi-liquid solution, and comprises one or more food excipients or one or more food ingredients for enhancing nutritional properties or organoleptic characteristics.

12. The food according to claim 11, wherein one liter of said liquid or semi-liquid solution comprises dried parts of at least one of the following plants:
guayusa: from 5 to 40 g/l;
guarana: from 2 to 20 g/l;
yerba mate: from 3 to 20 g/l;
and one of the following sweeteners;
dextrose: from 0 to 30 g/l;
sucralose: from 0 to 250 mg/l; or
stevioside or rebaudioside A: from 0 to 700 mg/l.

13. The food according to claim 1, wherein said food is obtained by mixing the composition made according to claim 1 into a liquid or semi-liquid solution comprising vegetable extracts of plants containing flavonoids, saponins, or amino acids.

14. The food according to claim 13, wherein said dilutable composition and said liquid and said liquid or semi-liquid solution are stored within separate containers up to ingestion, and wherein the liquid or semi-liquid solution is stored within a container closable by a storage cap within which the dilutable composition is stored.

15.-17. (canceled)

18. The food according to claim 1, wherein said food is provided within a kit comprising:
at least one container containing a beverage obtained by mixing the composition with a liquid or semi-liquid solution comprising vegetable extracts of plants containing flavonoids, saponins, or amino acids; and
means for controlling and monitoring effects resulting from an ingestion of the composition or of the liquid or semi-liquid solution.

19. The food according to claim 18, wherein the at least one container comprises:
at least one container containing the composition in dilutable form, and
at least one container containing the liquid or semi-liquid solution.

20. The food according to claim 19, wherein said means controlling and monitoring effects comprise a software executing tests controlling and monitoring nervous responsiveness, cognitive processes, or physical and mental performance.

21. A method of producing a liquid or semi-liquid solution comprising vegetable extracts according to claim 10, the solution being used as a beverage or for producing a beverage for human consumption, comprising the following steps:
selecting at least one plant containing desired active principles or preparing the beverage, said plant being selected from the group consisting of guayusa (Ilex guayusa), guarana (Paullinia cubana) or yerba mate (Ilex paraguariensis);
drying the plant under a controlled atmosphere;
grinding the dried plant;
extracting the active principles from the dried plant by ultrasound extraction machines by using a solvent; and
mixing liquid solutions obtained from each plant.

22. The method according to claim 21, further comprising a spraying and drying step causing reduction in powder of each liquid containing the active principles obtained from each plant or of a mixture of said liquids with a spray-dryer.

23. The method according to claim 21, wherein each liquid containing the active principles obtained from each plant or a mixture of said liquids is frozen at a temperature lower than 0.

24. The method according to claim 21, further comprising the step of mixing each liquid containing the active principles obtained from each plant or a mixture of said liquids, with a liquid or semi-liquid substance.
